(19) United States
(12) Patent Application Publication
(10) Pub. No.: US 2016/0166577 A1
(43) Pub. Date: Jun. 16, 2016

(54) TREATMENT OF PULMONARY FIBROSIS USING AN INHIBITOR OF CBP/CATENIN

(71) Applicant: PRISM Pharma Co., Ltd., Kanagawa (JP)

(72) Inventors: Hiroyuki KOUJI, Kanagawa (JP); Takenao ODAGAMI, Kanagawa (JP)

(73) Assignee: PRISM PHARMA CO., LTD., Kanagawa (JP)

(21) Appl. No.: 14/436,653

(22) PCT Filed: Oct. 21, 2013
(86) PCT No.: PCT/JP2013/079056

§ 371 Date: Apr. 17, 2015

Related U.S. Application Data
(60) Provisional application No. 61/716,069, filed on Oct. 19, 2012.

(51) Int. Cl. A61K 31/53

(52) U.S. Cl. CPC A61K 31/53

(57) ABSTRACT

The present disclosure relates generally to alpha-helix mimetic structures and specifically to alpha-helix mimetic structures that are inhibitors of β-catenin. The disclosure also relates to applications in the treatment of pulmonary fibrosis, including usual interstitial pneumonia and idiopathic pulmonary fibrosis, and pharmaceutical compositions comprising such alpha helix mimetic β-catenin inhibitors.

### CROSS-REFERENCE TO RELATED APPLICATIONS

This application claims priority to U.S. provisional application 61/716,069, filed Oct. 19, 2012, which is incorporated herein in its entirety.

### BACKGROUND OF THE DISCLOSURE

Wnt/β-catenin signaling is emerging as a forerunner for its critical roles in many facets of human biology. This signaling pathway has roles in embryogenesis, organogenesis, and maintaining tissue and organ homeostasis, and also in pathological conditions such as cancer and other human disorders such as inflammatory disorders and fibrosis. It is also integral in several physiological events such as differentiation, proliferation, survival, oxidative stress, morphogenesis, and others.

Idiopathic pulmonary fibrosis (IPF) and usual interstitial pneumonia (UIP) are ravaging conditions of progressive lung scarring and destruction. Anti-inflammatory therapies including corticosteroids have limited efficacy in this ultimately fatal disorder. Treatment options for pulmonary fibrosis are very limited. An important unmet need is to identify new agents that interact with key molecular pathways involved in the pathogenesis of pulmonary fibrosis to prevent progression or reverse fibrosis in these patients. Aberrant activation of the Wnt/β-catenin signaling cascade occurs in lungs of patients with IPF. This pathway has been targeted for intervention in pulmonary fibrosis using ICG-001, a small molecule that specifically inhibits T-cell factor/β-catenin transcription in a cyclic AMP response-element binding protein binding protein (CBP)-dependent fashion (PNAS 107:14309-14314, 2010). ICG-001 selectively blocks the β-catenin/CBP interaction without interfering with the β-catenin/p300 interaction. ICG-001 (5 mg/kg per day) significantly inhibits β-catenin signaling and attenuates bleomycin-induced lung fibrosis in mice, while concurrently preserving the epithelium. Administration of ICG-001 concurrent with bleomycin prevents fibrosis, and late administration is able to reverse established fibrosis and significantly improve survival. Because no effective treatment for IPF exists, selective inhibition of Wnt/β-catenin-dependent transcription suggests a potential unique therapeutic approach for pulmonary fibrosis.

### BRIEF SUMMARY OF THE DISCLOSURE

This disclosure presents methods of treating pulmonary fibrosis, including idiopathic pulmonary fibrosis, by administration of an inhibitor of β-catenin. This disclosure also provides alpha helix mimetic β-catenin inhibitor compounds, and compositions comprising an inhibitor of β-catenin.

### BRIEF DESCRIPTION OF THE FIGURES

FIGS. 1A-1B. Compound A reduces lung fibrosis (A) and collagen deposition (B) in bleomycin-treated mice. (A), Ashcroft score shows increased fibrosis in bleomycin (Bleo) treated mice relative to untreated (Ns) mice, which is reduced by treatment with Compound A (Bleo+Cmpd A). (B), Collagen assay shows increased collagen deposition (μg/lung) in bleomycin (Bleo) treated mice relative to untreated (Ns) mice, which is reduced by treatment with Compound A (Bleo+Cmpd A).

FIGS. 2A-2C. Histological examination shows that Compound A reduces lung fibrosis in bleomycin-treated mice. (A-C), Scale bar, 200 μM. (A), Normal lung in untreated mice shows absence of fibrotic (dark staining) tissue. (B), Bleomycin treatment induces substantial fibrotic changes as indicated by extensive areas of dark staining fibrotic tissue. (C), Compound A treatment reduces fibrosis in bleomycin-treated mice as indicated by reduced areas of dark-staining fibrotic tissue.

FIG. 3. Antifibrotic effects of Compound A on bleomycin-induced pulmonary fibrosis between the early and late treatment schedules. Bleo, bleomycin treatment alone; Bleo+Cmpd A whole, bleomycin plus Compound A administered throughout 4 week period; Bleo+early 2w, bleomycin plus Compound A administered over weeks 1-2; Bleo+late 2w, bleomycin plus Compound A administered over weeks 3-4.

FIG. 4. Antifibrotic effects of Compound A and ICG-001 on bleomycin-induced pulmonary fibrosis. Bleo, bleomycin treatment alone; Bleo+Cmpd A late 3w, bleomycin plus Compound A administered over weeks 4-6; Bleo+ICG late 3 w, bleomycin plus ICG-001 administered over weeks 4-6.

FIG. 5. Compound C inhibits epithelial-to-mesenchymal transition (EMT) induced by TGF-β treatment in the lung epithelial cell line LA-4. Increasing doses of Compound C inhibit TGF-β-induced alpha smooth muscle actin (α-SMA) formation in lung epithelial cells.

FIG. 6A-6D. Compound C inhibits epithelial-to-mesenchymal transition (EMT) induced by TGF-β treatment in the lung epithelial cell line LA-4. Fluorescent microscopy of LA-4 cells treated with TGF-β 5 ng/ml (A, C) or TGF-β 5 ng/ml plus Compound C 0.01 μM (B, D) shows decreased alpha smooth muscle actin (α-SMA) expression in cells treated with TGF-β plus Compound C (D) relative to TGF-β alone (B), while DAPI staining identifies cell nuclei in both TGF-β (A) and TGF-β plus Compound C cells (B).

### DETAILED DESCRIPTION OF THE DISCLOSURE

Recently, non-peptide compounds have been developed which mimic the secondary structure of reverse-turns found in biologically active proteins or peptides. For example, U.S. Pat. No. 5,440,013 and published PCT Applications Nos. WO94/03494, WO01/00210A1, and WO01/16135A2 each disclose conformationally constrained, non-peptidic compounds, which mimic the three-dimensional structure of reverse-turns. In addition, U.S. Pat. No. 5,929,237 and its continuation-in-part U.S. Pat. No. 6,013,458, disclose conformationally constrained compounds which mimic the secondary structure of reverse-turn regions of biologically active peptides and proteins. In relation to reverse-turn mimetics, conformationally constrained compounds have been disclosed which mimic the secondary structure of alpha-helix regions of biologically active peptide and proteins in WO2007/056513 and WO2007/056593.

This disclosure provides novel compounds, pharmaceutical compositions and methods of treatment for pulmonary fibrosis. The inventors have determined that inhibiting β-catenin signaling is an effective approach to the treatment of fibrotic lung diseases.

The structures and compounds of the alpha helix mimetic β-catenin inhibitors of this invention are disclosed in WO 2010/044485, WO 2010/128685, WO 2009/148192, and US 2011/0092459, each of which is incorporated herein by reference in its entirety. These compounds have now been found to be useful in the treatment of pulmonary fibrosis.

The preferable structure of the alpha helix mimetic β-catenin inhibitors of this invention have the following formula (I):

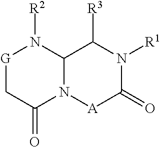

wherein

### A is —CHR⁷—,

wherein

R⁷ is optionally substituted arylalkyl, optionally substituted heteroarylalkyl, optionally substituted cycloalkylalkyl or optionally substituted heterocycloalkylalkyl;

### G is —NH—, —NR⁶—, or —O—

wherein

R⁶ is lower alkyl or lower alkenyl;

R¹ is —Ra—R¹⁰;

wherein

Ra is optionally substituted lower alkylene and

R¹⁰ is optionally substituted bicyclic fused aryl or optionally substituted bicyclic fused heteroaryl;

R² is —(CO)—NH—Rb—R²⁰,

wherein

Rb is bond or optionally substituted lower alkylene; and

R²⁰ is optionally substituted aryl or optionally substituted heteroaryl; and

R³ is C₁₋₄ alkyl.
These compounds are especially useful in the prevention and/or treatment of pulmonary fibrosis.

The more preferable structure of the alpha helix mimetic β-catenin inhibitors of this invention have the following substituents in the above-mentioned formula (I):

### A is —CHR⁷—,

wherein

R⁷ is arylalkyl optionally substituted with hydroxyl or C₁₋₄ alkyl;

### G is —NH—, —NR⁶—, or —O—

wherein

R⁶ is C₁₋₄ alkyl or C₁₋₄ alkenyl;

R¹ is —Ra—R¹⁰;

wherein

Ra is C₁₋₄ alkylene and

R¹⁰ is bicyclic fused aryl or bicyclic fused heteroaryl, optionally substituted with halogen or amino;

R² is —(CO)—NH—Rb—R²⁰,

wherein

Rb is bond or C₁-4 alkylene; and

R²⁰ is aryl or heteroaryl; and

R³ is C₁₋₄ alkyl.
These compounds are especially useful in the prevention and/or treatment of pulmonary fibrosis.

The most preferable alpha helix mimetic β-catenin inhibitors of this invention are as follows:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.
   These compounds are especially useful in the prevention and/or treatment of pulmonary fibrosis.

In a most preferred embodiment, the compound is:
4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate, or
(6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.
   These compounds are especially useful in the prevention and/or treatment of pulmonary fibrosis.

While not wishing to be bound, the effectiveness of these compounds in treating these conditions is based in part on the ability of these compounds to block TCF4/β-catenin transcriptional pathway by inhibiting cyclic AMP response-element binding protein (CBP), thus altering wnt pathway signaling, which has been found to improve outcomes.

A “β-catenin inhibitor” is a substance that can reduce or prevent β-catenin activity. β-catenin activities include translocation to the nucleus, binding with TCF (T cell factor) transcription factors, and coactivating TCF transcription factor-induced transcription of TCF target genes. A “β-catenin inhibitor” can also interfere with the interaction of CBP and β-catenin. Thus, a β-catenin inhibitor inhibits or reduces CBP/β-catenin signaling and activity of the CBP/β-catenin signaling pathway, including reduction of one or more downstream signaling events.

Disclosed herein are alpha helix mimetic β-catenin inhibitor compounds for treatment of pulmonary fibrosis.

### Diseases

As used herein, “treatment” refers to clinical intervention in an attempt to alter the disease course of the individual or cell being treated, and can be performed during the course of clinical pathology. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis.

As used herein, the terms “therapeutically effective amount” and “effective amount” are used interchangeably to refer to an amount of a composition of the invention that is sufficient to result in the prevention of the development or onset of pulmonary fibrosis, or one or more symptoms thereof, to enhance or improve the effect(s) of another therapy, and/or to ameliorate one or more symptoms of pulmonary fibrosis. For a subject suffering from pulmonary fibrosis, a preferred therapeutically effective amount is an amount effective to reduce fibrosis and/or improve lung function.

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

The β-catenin inhibitors described herein are useful to prevent or treat disease. Specifically, the disclosure provides for both prophylactic and therapeutic methods of treating a subject at risk of (or susceptible to) pulmonary fibrosis. Accordingly, the present methods provide for the prevention and/or treatment of pulmonary fibrosis in a subject by administering an effective amount of a β-catenin inhibitor to a subject in need thereof. For example, a subject can be administered the β-catenin inhibitor composition in an effort to improve one or more of the factors of a pulmonary fibrosis condition.

As used herein, “pulmonary fibrosis” is defined as excessive accumulation of connective or scar tissue within the lung. The accumulation of connective/scar tissue in pulmonary fibrosis is excessive compared to connective tissue levels in a normal, healthy lung. This fibrosis is often accompanied by necrosis and/or inflammation of lung tissue. β-catenin signaling plays a role in inducing the over-production and excess accumulation of an extracellular matrix such as collagen.

Pulmonary fibrosis can be a secondary effect of connective tissue diseases caused by autoimmune disorders, inhalation of environmental and occupational pollutants, viral infections, or other interstitial lung diseases which cause injuries to the lung. If the cause of the pulmonary fibrosis is known, it is classified as usual interstitial pneumonia (UIP). If the cause is unknown, idiopathic pulmonary fibrosis (IPF) or idiopathic interstitial pneumonia (IIP) is diagnosed.

The invention also encompasses methods where the compound is given in combination therapy. That is, the compound can be used in conjunction with, but separately from, other agents useful in treating pulmonary fibrosis. In these combination methods, the compound will generally be given in a daily dose of 1-100 mg/kg body weight daily in conjunction with other agents. The other agents generally will be given in the amounts used therapeutically. The specific dosing regime, however, will be determined by a physician using sound medical judgment.

Treatment of pulmonary fibrosis refers to the administration of a compound or combination described herein to treat a subject suffering from pulmonary fibrosis. One outcome of the treatment of pulmonary fibrosis is to reduce formation of excessive connective tissue. Another outcome of the treatment of pulmonary fibrosis is to reduce inflammation and infiltration of immune cells. Still another outcome of the treatment of pulmonary fibrosis is to reduce lung tissue necrosis. Still another outcome of the treatment of pulmonary fibrosis is to improve lung function.

The alpha helix mimetic β-catenin inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Any suitable route of administration may be employed for providing a mammal, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The effective dosage of active ingredient employed may vary depending on the particular compound employed, the mode of administration, the condition being treated and the severity of the condition being treated. Such dosage may be ascertained readily by a person skilled in the art.

When treating or controlling pulmonary fibrosis and/or other diseases for which compounds described herein are indicated, generally satisfactory results are obtained when the compounds described herein are administered at a daily dosage of from about 0.01 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

Also described herein are pharmaceutical compositions which comprise a compound described herein and a pharmaceutically acceptable carrier. The pharmaceutical compositions described herein comprise a compound described herein or a pharmaceutically acceptable salt as an active ingredient, as well as a pharmaceutically acceptable carrier and optionally other therapeutic ingredients. A pharmaceutical composition may also comprise a prodrug, or a pharmaceutically acceptable salt thereof, if a prodrug is administered.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds described herein may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The present disclosure is further illustrated by the following non-limiting examples.

### EXAMPLES

### Bleomycin (BLM)-Induced Pulmonary Fibrosis in Mice

C57BL/6 mice received subcutaneous implants of an ALZET mini-osmotic pump (Durect Corp., California) that released BLM continuously (Example 1, 140 mg/kg/mouse/day; Example 2, 120 U/kg/mouse/day) for seven days (Day 1-Day 7), to induce lung fibrosis (Lab Invest. 50:487-488, 1984; Am. J. Resp. Cell Mol. Biol. 45:489-497, 2011). Bleomycin induces epithelial injury, causing increased pulmonary inflammation from Days 1-21, peaking at about Day 10 before subsiding. Fibrosis begins at about Day 8 and gradually increases until Day 28.

Test Compound.

BLM mice treated with test compound received a second subcutaneous ALZET mini-osmotic pump (Durect Corp., California) that released Compound A continuously. Compound A, 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate.

Assessment of Fibrosis.

Ashcroft score (J. Clin. Pathol. 41:467-470, 1988) was calculated from tissue sections of right lung stained by H&E. Left lung homogenates were analyzed for soluble collagen content using SIRCOL Collagen Assay kit (Biocolor Ltd., Carrickfergus, UK).

### Example 1

In Example 1, Compound A was administered 5 mg/kg/day over days 15-28.

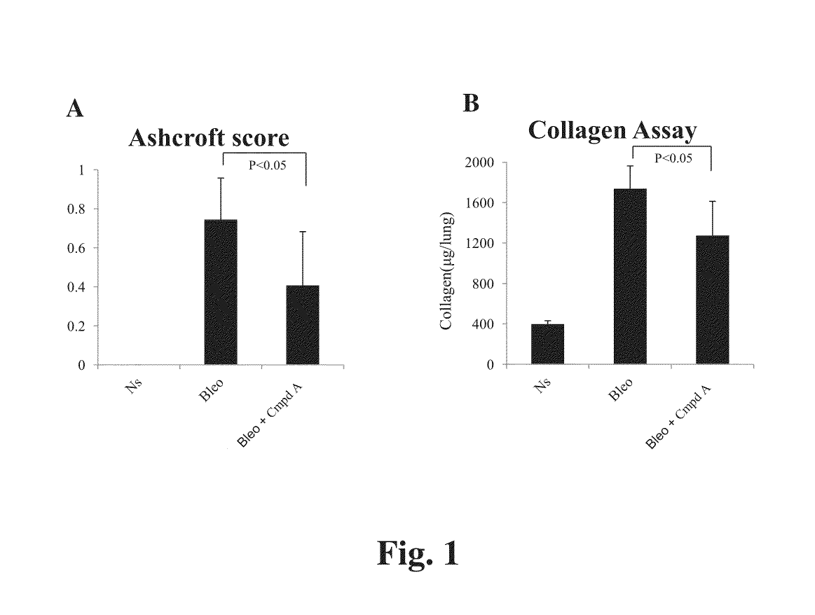

As seen in FIGS. 1A-1B, Compound A (5 mg/kg, days 14-28) reduces lung fibrosis (FIG. 1A) and collagen deposition (FIG. 1B) in bleomycin-treated mice. (FIG. 1A), Ashcroft score shows increased fibrosis in bleomycin (Bleo) treated mice relative to untreated (Ns) mice, which is reduced by treatment with Compound A (Bleo+Cmpd A). (FIG. 1B), Collagen assay shows increased collagen deposition (μg/lung) in bleomycin (Bleo) treated mice relative to untreated (Ns) mice. Collagen deposition in BLM mice is reduced by treatment with Compound A (Bleo+Cmpd A).

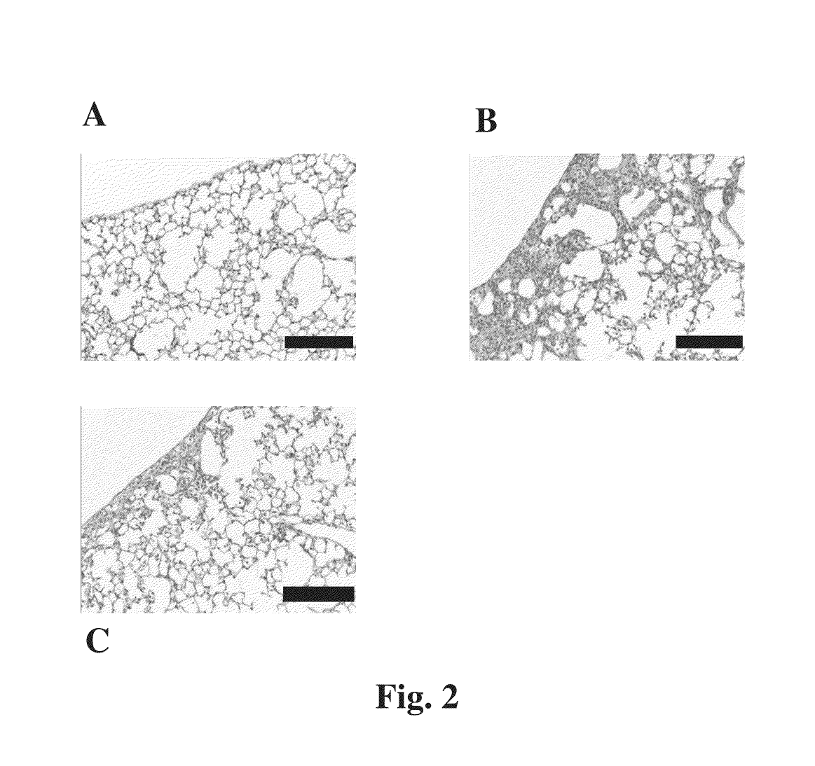

As seen in FIGS. 2A-2C, histological examination shows that Compound A (5 mg/kg, days 14-28) reduces lung fibrosis in bleomycin-treated mice. (FIG. 2A), Normal lung in untreated mice shows absence of fibrotic (dark staining) tissue. (FIG. 2B), bleomycin treatment induces substantial fibrotic changes as indicated by extensive areas of dark staining fibrotic tissue. (FIG. 2C), Compound A treatment reduces fibrosis in bleomycin-treated mice, as indicated by reduced areas of dark-staining fibrotic tissue.

### Example 2

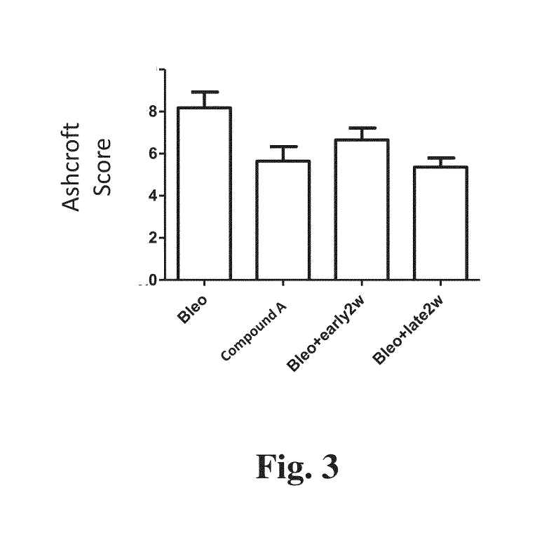

In Example 2, Compound A was administered 1 mg/kg/day over Days 1-28 (“whole”), Days 1-14 (“early”) or Days 15-28 (“late”). As seen in FIG. 3, Compound A has an antifibrotic effect on bleomycin-induced pulmonary fibrosis between the early and late treatment schedules. Bleo, bleomycin treatment alone; Bleo+Cmpd A whole, bleomycin plus Compound A administered throughout 4 week period; Bleo+early 2w, bleomycin plus Compound A administered over weeks 1-2; Bleo+late 2w, bleomycin plus Compound A administered over weeks 3-4.

Compound A administered only during the early (inflammatory) phase was not significantly beneficial over no test compound at all. In contrast, Compound A administered during the late (fibrotic) phase of lung fibrosis development was equal to, or even superior to, administration of Compound A throughout the entire period, and significantly reduced lung fibrosis.

### Example 3

In Example 3, a six-week rather than four-week course of BLM-induced fibrosis was studied. In this example, either Compound A (5 mg/kg/day) or ICG-001 (5 mg/kg/day), another β-catenin inhibitor, was administered to BLM-treated mice over weeks 4-6.

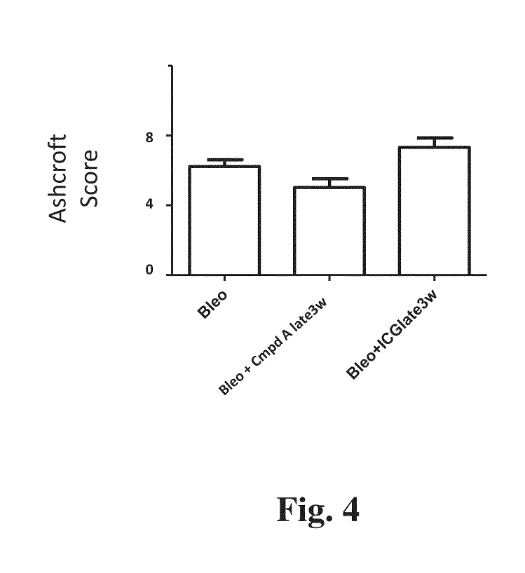

As seen in FIG. 4, Compound A has an antifibrotic effect, unlike β-catenin inhibitor ICG-001, on bleomycin-induced pulmonary fibrosis. Bleo, bleomycin treatment alone; Bleo+Cmpd A late 3w, bleomycin plus Compound A administered over weeks 4-6; Bleo+ICG late 3 w, bleomycin plus ICG-001 administered over weeks 4-6.

### Example 4

In Example 4, the murine lung epithelial cell line, LA-4, available from the American Type Culture Collection (ATCC; Manassas, Va.), was treated with TGF-β (5 ng/ml) to induce epithelial-to-mesenchymal transition (EMT), a marker for fibrosis (J Clin Invest. 119:1420-1428, 2009). TGF-β treated cells were treated with no or with increasing concentrations (0.01 μM, 0.05 μM, 0.1 μM, or 0.5 μM) of Compound C. Compound C, (6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

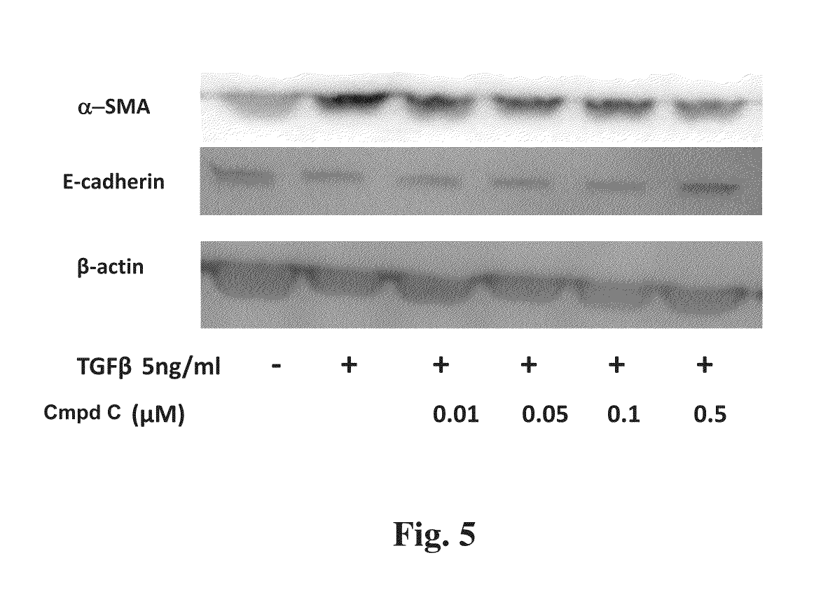

As seen in FIG. 5, Compound C inhibits epithelial-to-mesenchymal transition (EMT) induced by TGF-β treatment in the lung epithelial cell line LA-4. Increasing doses of Compound C inhibit TGF-β-induced alpha smooth muscle actin (α-SMA) formation in lung epithelial cells.

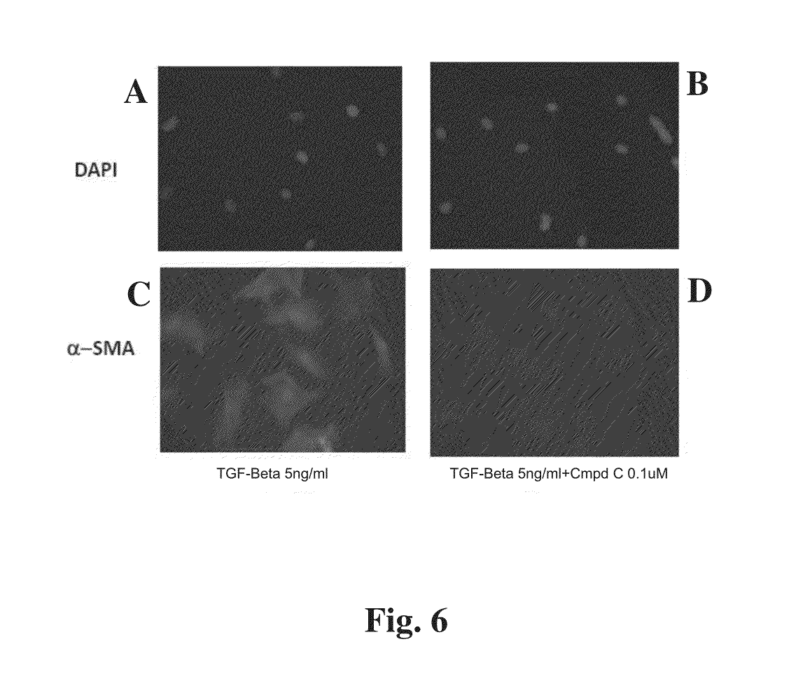

As seen in FIGS. 6A-6D, Compound C inhibits epithelial-to-mesenchymal transition (EMT) induced by TGF-β treatment in the lung epithelial cell line LA-4. Fluorescent microscopy of LA-4 cells treated with TGF-β 5 ng/ml (A, C) or TGF-β 5 ng/ml plus Compound C 0.01 μM (B, D) shows decreased alpha smooth muscle actin (α-SMA) expression in cells treated with TGF-β plus Compound C (D) relative to TGF-β alone (B), while DAPI staining identifies cell nuclei in both TGF-β (A) and TGF-β plus Compound C cells (B).

1. An alpha helix mimetic β-catenin inhibitor compound for the treatment of pulmonary fibrosis, having the following formula (I): 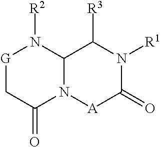
wherein:
A is —CHR⁷—,
wherein R⁷ is hydrogen, optionally substituted alkyl, optionally substituted alkenyl, optionally substituted alkynyl, optionally substituted arylalkyl, optionally substituted heteroarylalkyl, optionally substituted cycloalkylalkyl, optionally substituted heterocycloalkylalkyl, optionally substituted aryl, optionally substituted heteroaryl, optionally substituted cycloalkyl or optionally substituted heterocycloalkyl;
G is —NH—, —NR⁶—, —O—, —CHR⁶— or —C(R⁶)₂—,
wherein R⁶ is independently selected from optionally substituted alkyl, optionally substituted alkenyl and optionally substituted alkynyl;
R¹ is optionally substituted arylalkyl, optionally substituted heteroarylalkyl, optionally substituted cycloalkylalkyl or optionally substituted heterocycloalkylalkyl;
R² is W²¹—W²²—Rb—R²⁰,
wherein W²¹ is —(CO)— or —(SO₂)—; W²² is bond, —O—, —NH— or optionally substituted lower alkylene; Rb is bond or optionally substituted lower alkylene; and R²⁰ is optionally substituted alkyl, optionally substituted alkenyl, optionally substituted alkynyl, optionally substituted aryl, optionally substituted heteroaryl, optionally substituted cycloalkyl or optionally substituted heterocycloalkyl; and
R³ is optionally substituted alkyl, optionally substituted alkenyl or optionally substituted alkynyl;
or a pharmaceutically acceptable salt thereof.

2. The compound of claim 1, selected from:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.

3. The compound of claim 1, selected from:
4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
or (6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

4. A pharmaceutical composition comprising the compound of claim 1.

5. A method of treatment for pulmonary fibrosis, comprising administering an effective amount of the compound of claim 1 to a patient in need thereof.

6. The method of claim 5, wherein the pulmonary fibrosis is usual interstitial pneumonia.

7. The method of claim 5, wherein the pulmonary fibrosis is idiopathic pulmonary fibrosis.
